(19) United States
(12) Patent Application Publication
(10) Pub. No.: US 2024/0122832 A1
(43) Pub. Date: Apr. 18, 2024

(54) SUBSTITUTION OF AMMONIA IN HAIR ALTERING PRODUCTS

(71) Applicant: Angus Chemical Company, Buffalo Grove, IL (US)

(72) Inventors: Mrunalini Shireesh Dhamdhere, Vernon Hills, IL (US); Ruth Keiko Kuriyama, Sao Paulo (BR); Bonnie A. Pyzowski, Park Ridge, IL (US)

(21) Appl. No.: 18/278,299

(22) PCT Filed: Feb. 18, 2022
(86) PCT No.: PCT/US2022/017063

§ 371 Date: Aug. 22, 2023

Related U.S. Application Data
(60) Provisional application No. 63/152,544, filed on Feb. 23, 2021.

(51) Int. Cl. A61K 8/41; A61K 8/22; A61K 8/23; A61K 8/34; A61K 8/44; A61K 8/67; A61K 8/92; A61Q 5/04; A61Q 5/10

(52) U.S. Cl. CPC A61K 8/41; A61K 8/22; A61K 8/23; A61K 8/342; A61K 8/442; A61K 8/676; A61K 8/922; A61Q 5/04; A61Q 5/10; A61K 2800/522

(57) ABSTRACT

The present technology provides hair-altering compositions that include 2-aminopropanol (DL-2AP). The present technology also provides kits that include the hair-altering composition and methods of using the hair-altering composition.

### CROSS-REFERENCE TO RELATED APPLICATIONS

This application claims the benefit of priority to U.S. Provisional Patent Application No. 63/152,544 filed Feb. 23, 2021, which is hereby incorporated by reference, in its entirety for any and all purposes.

### FIELD OF THE INVENTION

The invention relates to a hair altering compositions and processes using the compositions.

### BACKGROUND OF THE INVENTION

Hair altering compositions commonly include ammonia to adjust the composition's pH and/or open hair cuticles. For example, permanent hair color altering compositions typically include two compositions mixed just prior to hair application. The first composition includes ammonia and often one or more colorants (e.g., dyes, pigments, and/or coupling agents) and the second composition, often referred to as a developer, includes an oxidizing agent (e.g., peroxide such as hydrogen peroxide). The ammonia opens the cuticle layer of the hair, so the developer and colorants can together penetrate the hair cortex. To achieve the final colorant, commonly the dye/pigment is oxidized by the oxidizing agent, reacts with a coupling agent, and then may be further oxidized (i.e., oxidative condensation reaction). The oxidative condensation reaction is often completed within the cortex to provide large sized colorant complexes trapped inside the hair fiber to provide permanent hair color alteration. For a dark hair color to be altered to a lighter hair color, commonly higher concentrations of the developer are needed to lift the color of the hair and deposit the colorants. To achieve semi-permanent color alteration (i.e., resists removal of the dyes/pigments from the hair over several cycles of shampoo washing), the dyes/pigments are often not chemically modified by oxidative condensation such that the dyes/pigments in the colorant are directly used for altering the hair color, but are significantly less likely to be trapped within the cortex due to the difficulty in penetrating the cortex due to their large size. Semi-permanent color altering compositions may or may not include an oxidizing agent.

Ammonia has a pungent smell. In higher concentrations, it can be caustic and hazardous, and at even low concentrations, it can cause skin irritation. There is a need for hair altering compositions having the appropriate pH and/or that are capable of opening the cuticle layer of the hair without the drawbacks of ammonia.

### SUMMARY OF THE INVENTION

The present application provides hair-altering compositions that include an alkaline agent, which includes 2-aminopropan-1-ol, (also referred to herein as 2-aminopropanol or DL-2AP, but which is not limited to racemic mixtures of enantiomers). In any embodiment, the compositions may include about 2 wt % to about 15 wt % including about 3 wt % to about 14 wt %, about 3 wt % to about 10 wt %, or about 6 wt % to about 11 wt % of the DL-2AP. In any embodiment, the compositions have significantly lower odor compared to a composition that differs only by substituting the DL-2AP with the same amount of ammonia. In any embodiment, the composition may be for altering curl, kink, texture, and/or color of hair. In any embodiment, the composition may be for altering curl, kink, and/or texture of hair (e.g., relaxation of naturally curly or kinky hair or curling naturally straight hair). In any embodiment, the composition may be for altering color of hair. In any embodiment, hair treated with the composition may exhibit a hair color altering ΔE of less than about 2.5 compared to hair treated with a control composition having ammonia substituted for the DL-2AP (i.e., the DL-2AP composition treated hair and the ammonia composition treated hair have a ΔE of less than about 2.5). In any embodiment, hair treated with the composition may exhibit a cysteic acid difference of less than about 40% compared to a control composition having ammonia substituted for the 2-aminopropanol.

The present application also provides kits including the hair-altering composition. The kit may include a first composition that includes the DL-2AP and a second composition that includes a developer. Following combination of the first composition and the second composition, the hair-altering compositions disclosed herein are achieved.

In another aspect, the present application provides a method for altering hair including applying the composition disclosed herein on a hair; and rinsing the composition off of the hair with water to provide the altered hair.

### BRIEF DESCRIPTION OF THE DRAWINGS

FIG. 1 is a graph illustrating lightening or bleaching power via color tone change.

FIGS. 2A and 2B are graphs illustrating the accumulative protein loss indicated by the increase of cysteic acid of hair fibers measured by FTIR-ATR at 7 wt % DL-2AP (FIG. 2A) or 11 wt % DL-2AP (FIG. 2B), according to the examples.

FIG. 3 are photos hair samples illustrating blond color deposition and fastness.

FIG. 4 are photos of hair samples illustrating dark brown color deposition and fastness.

FIG. 5 are photos of hair samples illustrating red color deposition and fastness.

FIG. 6 is a graph illustrating color wash fastness differences between hair samples treated with blond color deposition comprising ammonium, MEA, and DL-2AP, respectively.

FIG. 7 are photos of a graph illustrating color wash fastness differences between hair samples treated with dark brown color deposition comprising ammonium, MEA, and DL-2AP (5% and 3%), respectively.

FIG. 8 are photos of a graph illustrating color wash fastness differences between hair samples treated with red color deposition comprising ammonium, MEA, and DL-2AP, respectively.

FIGS. 9A-9D are photos of hair samples after treatment with a straightening composition including ammonia, DL-2AP, MEA, AMP, or AMPD, according to the examples.

FIG. 10 are photos of hair samples illustrating gray hair coverage and color deposition and fastness.

FIG. 11 is a graph illustrating color wash fastness differences between hair samples in FIG. 10 treated with hair deposition comprising ammonium, MEA, and DL-2AP (5% and 3%), respectively.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

In general, “substituted group” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In any embodiment, a substituted group is substituted with 1, 2, 3, 4, 5, or 6 substituents. Examples of substituent groups include: halogens (i.e., F, Cl, Br, and I); CF₃; hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; amines; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted

The term “alkyl” refers to a group, whether alone or as part of another group (e.g., in dialkylamino), encompasses straight and branched chain aliphatic groups (i.e., saturated hydrocarbyl chains), and, unless otherwise indicated, has 1-10, alternatively 1-8, or alternatively 1-6 alkyl carbon atoms. Preferred alkyl groups include, without limitation, methyl, ethyl, propyl, isopropyl, butyl, isobutyl, sec-butyl, tert-butyl, pentyl, and hexyl. Unless otherwise indicated, the alkyl group is optionally substituted with 1, 2, or 3, preferably 1 or 2, more preferably 1, substituents that are compatible with the compounds, monomers, and polymers described herein. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like. In any embodiment, the alkyl group is unsubstituted.

Alkenyl groups include straight and branched chain alkyl groups as defined above, except that at least one double bond exists between two carbon atoms. Alkenyl groups have from 2 to 12 carbon atoms, and typically from 2 to 10 carbons or, In any embodiment, from 2 to 8, 2 to 6, or 2 to 4 carbon atoms. In any embodiment, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

The term “(hetero)cycloalkyl” refers to cycloalkyl and heterocycloalkyl groups.

The term “cycloalkyl” refers to saturated cyclic hydrocarbon groups. Unless otherwise indicated, the cycloalkyl group has 3 to 12 ring carbon atoms, alternatively 3 to 8 ring carbon atoms, or alternatively 3 to 6 ring carbon atoms. Preferred cycloalkyl groups include, without limitation, cyclopropyl, cyclobutyl, cyclopentyl, cyclopentenyl, and cyclohexyl. Unless otherwise indicated, the cycloalkyl group is optionally substituted with 1, 2, or 3, preferably 1 or 2, more preferably 1 alkyl group. In any embodiment, the alkyl group may include 1-6 carbon atoms, preferably the alkyl group is unsubstituted and includes 1-4 carbon atoms. In any embodiment, the cycloalkyl group is unsubstituted.

The term “heterocycloalkyl” as used herein refers to non-aromatic ring compounds containing 5 or more ring members, of which at least three are carbon atoms and at least one is a nitrogen atom. In any embodiment, the heterocyclyl group contains 1 or 2 heteroatoms. In any embodiment, the heterocyclyl group may include at least 4 or at least 5 carbon atoms. Typically, the heterocycloalkyl group is unsubstituted.

The term “(hetero)aryl” refers to aryl and heteroaryl groups.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In any embodiment, aryl groups contain 6-14 carbons, and in others from 6 to 12 or even 6-10 carbon atoms in the ring portions of the groups. In any embodiment, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group or its ionized form —COO—.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In any embodiment, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In any embodiment, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “halogen” or “halo” as used herein refers to bromine, chlorine, fluorine, or iodine. In any embodiment, the halogen is fluorine. In other embodiments, the halogen is chlorine or bromine.

The term “hydroxyl” as used herein can refer to —OH or its ionized form, —O—. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

Groups described herein having two or more points of attachment (i.e., divalent, trivalent, or polyvalent) within the compound of the present technology are designated by use of the suffix, “ene.” For example, divalent alkyl groups are alkylene groups, divalent cycloalkyl groups are cycloalkylene groups, and so forth. Substituted groups having a single point of attachment to the compound of the present technology are not referred to using the “ene” designation. Thus, e.g., chloroethyl is not referred to herein as chloroethylene.

As used herein, “substantially free” refers to less than about 2 wt % of the specified component based on the total weight of the composition. In any embodiment, the composition may include less than about 1 wt %, less than about 0.5 wt %, or less than about 0.1 wt %. In any embodiment, the composition may free of detectable amounts of the component.

As used herein, “ΔE” is calculated using the following equation:

ΔE_{ab}*=(L₂*−L₁*)²+(a₂*−a₁*)²+(b₂*−b₁*)²

The equation is according to the CIELAB color space also referred to as L*a*b* defined by the International Commission on Illumination (CIE) in 1976. It expresses color as three values: L* lightness value from 0 (black) to 100 (white), a* axis represents green-red component, and b* axis represents blue-yellow component. The calculation of ΔE is useful in industry for detecting small differences in color. Herein, the three-dimensional L*a*b* parameters may be measured using BYK Gardner Spectro-Guide Sphere. To compare hair treated with the composition including DL-2AP and hair treated with a control composition including ammonia rather than DL-2AP, the ΔE is provided herein. The control composition differs from the DL-2AP only by the ammonia replacing DL-2AP (about 1:1 mole substitution). For the color lifting test, the ΔE is determined between untreated virgin hair and color-treated hair. For the color deposition test, the ΔE was calculated for untreated hair and measurement after 5, 10, and 15 washes.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Ammonia is commonly used in hair altering compositions as an alkylating agent to open the cuticle layer of the hair. However, ammonia, while efficient at opening the cuticle layer, has a very strong odor and can be irritating to skin. As such, alternatives to ammonia in hair-altering compositions are in need. Monoethanolamine (MEA) is one known alternative due to its subtle odor. However, MEA does not open the cuticle layer as efficiently as ammonia leading to an unpredictable result and can cause skin irritation. The present technology provides hair-altering compositions that include an alkaline agent comprising 2-aminopropanol. In any embodiments, the 2-aminopropanol used in the alkaline agent is racemic. However, either enantiomer or mixtures of enantiomers may be used in the alkaline agent. The compositions of the present technology efficiently open the cuticle layer of hair.

In any embodiment, the 2-aminopropanol may be present in less than about 20 wt %. In any embodiment, the 2-aminopropanol may be present in an amount of about 2 wt % to about 15 wt %. In any embodiment, the 2-aminopropanol may be present in an amount of about 3 wt % to about 10 wt %. In any embodiment, the 2-aminopropanol may be present in an amount of about 3 wt % to about 14 wt %, about 4 wt % to about 12 wt %, about 5 wt % to about 12 wt %, about 6 wt % to about 12 wt %, about 5 wt % to about 11 wt %, or about 6 wt % to about 11 wt %.

In any embodiment, the composition may further include one or more alkanolamines different from 2-aminopropanol, ammonia, arginine, hydroxide based hair straighteners (e.g., guanidine hydroxide), or a combination of two or more thereof. In any embodiment, the composition may include ammonia. In any embodiment, the composition may include arginine. In any embodiment, the composition may include hydroxide based hair straighteners (e.g., guanidine hydroxide). In any embodiment, the composition may include one or more alkanolamines different from 2-aminopropanol, ammonia, arginine, hydroxide based hair straighteners (e.g., guanidine hydroxide), or a combination thereof. In any embodiment, the one or more alkanolamines may include 2-amino-2-methyl-1-propanol (AMP), 2-dimethylamino-2-methyl-1-propanol (DMAMP), 2-amino-1-butanol (AB), 2-amino-2-methyl-1,3-propanediol (AMPD), 2-amino-2-ethyl-1,3-propandiol (AEPD), tris(hydroxymethyl)aminomethane (TA), monoethanolamine (MEA), monoisopropanolamine (MIPA), triethanolamine (TEA) triisopropanolamine (TIPA), or combinations of two or more thereof. In any embodiment, the composition may include about 0.1 wt % to about 15 wt % of the one or more alkanolamines, ammonia, arginine, hydroxide based hair straighteners, or a combination thereof. In any embodiment, the composition may include about 0.5 wt % to about 12 wt % of the one or more alkanolamines, ammonia, arginine, hydroxide based hair straighteners, or a combination thereof. In any embodiment, the composition may include about 2 wt % to about 10 wt % of the one or more alkanolamines, ammonia, arginine, hydroxide based hair straighteners, or a combination thereof. In any embodiment, the composition may include about 3 wt % to about 10 wt % of the one or more alkanolamines, ammonia, arginine, hydroxide based hair straighteners, or a combination thereof. In any embodiment, the composition may include about 2 wt % to about 5 wt % of the one or more alkanolamines, ammonia, arginine, hydroxide based hair straighteners, or a combination thereof.

In other embodiments, the composition may be substantially free of or free of the one or more alkanolamines different from DL-2AP, ammonia, arginine, hydroxide based hair straighteners, or a combination of two or more thereof. In some embodiments, the composition may be substantially free of or free of ammonia. In some embodiments, the composition may be substantially free of or free of the one or more alkanolamines different from DL-2AP and ammonia.

In other embodiments, the composition may include a pH adjusting agent. In any embodiment, the pH adjusting agent may include tocopherol, glycerin phosphoric acid, phosphoric acid, sodium hydroxide, or combinations of two or more thereof.

In any embodiment, hair-altering composition may further include a coloring agent. For example, the composition may include any of the coloring agents provided in US Patent Appl. Publ. Nos. 2020/0289390, 2020/0360257, 2019/0365625, and 20140338135 and U.S. Pat. Nos. 8,343,238 and 6,818,022, which are each herein incorporated by reference. In any embodiment, the coloring agent may include oxidative dye or pigment precursors (including couplers such as meta-diamines, meta-aminophenols, meta-diphenols and certain heterocyclic compounds, such as indole compounds. In any embodiment, the coloring agent may include direct dyes or pigments. In any embodiment, the coloring agent may include both oxidative dye or pigment precursors and direct dyes or pigments. In any embodiment, the coloring agent may include anion dyes, cation dyes, neutral dyes, or combinations of two or more thereof.

Examples of anion dyes include, but are not limited to, Acid Black 1, Acid Blue 1, Acid Blue 3, Food Blue No. 2, Food Blue No. 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, Acid Orange 24, Acid Green 25, Solvent Green 7, Solvent Red 73, Acid Red 95, Solvent Red 43, Solvent Red 48, Acid Red 33, Solvent Violet 13, Acid Yellow 73, Food Red No. 17, Food Red No. 1, Food Yellow No. 3, Food Blue No. 2, Food Black No. 1, Food Black No. 2, Disperse Black 9, Disperse Violet 1, alkali metal salts thereof (sodium salt and potassium salt), or combinations of two or more thereof.

Examples of cation dyes include, but are not limited to, Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green 1, Basic Red 2, Basic Red 12, Basic Red 22, Basic Red 76, Basic Violet 1, Basic Violet 2, Basic Violet 3, Basic Violet 10, Basic Violet 14, Basic Yellow 57, Basic Red 51, Basic Yellow 87, Basic Blue 17, Basic Orange 31, or combinations of two or more thereof.

Examples of neutral dyes include, but are not limited to, nitro dyes include, HC Blue 2, HC Blue 4, HC Blue 5, HC Blue 6, HC Blue 7, HC Blue 8, HC Blue 9, HC Blue 10, HC Blue 11, HC Blue 12, HC Blue 13, HC Brown 1, HC Brown 2, HC Green 1, HC Orange 1, HC Orange 2, HC Orange 3, HC Orange 5, HC Red BN, HC Red 1, HC Red 3, HC Red 7, HC Red 8, HC Red 9, HC Red 10, HC Red 11, HC Red 13, HC Red 54, HC Red 14, HC Violet BS, HC Violet 1, HC Violet 2, HC Yellow 2, HC Yellow 4, HC Yellow 5, HC Yellow 6, HC Yellow 7, HC Yellow 8, HC Yellow 9, HC Yellow 10, HC Yellow 11, HC Yellow 12, HC Yellow 13, HC Yellow 14, HC Yellow 15, 2-amino-6-chloro-4-nitrophenol, picramic acid, 1,2-diamino-4-nitrobenzene, 1,4-diamino-2-nitrobenzene, 3-nitro-4-aminophenol, 1-hydroxy-2-amino-3-nitrobenzene, 2-hydroxyethylpicramic acid, 3-nitro-p-hydroxyethylaminophenol, 4-hydroxypropylamino-3-nitrophenol, and N,N-bis(2-hydroxyethyl)-2′-nitro-p-phenylenediamine, p-phenylenediamine, toluene-2,5-diamine, p-aminophenol, o-aminophenol, m-aminophenol, 4-aminometacresol, 1-hydroxyethyl-4,5-diaminopyrazole, 2,4-diaminophenoxyethanol, 2-methyl-5-aminophenol, resorcin, 2-methylresorcin, 4-chlororesorcinol, 1-naphthol, 2-amino-3-hydroxypyridine, 2-amino-4-(6-hydroxyethyl)aminoanisole, bis(phenyl)alkylenediamines, heterocyclic bases, salts thereof, or combinations of two or more thereof.

The p-phenylenediamine includes, for example, are p-phenylenediamine, p-toluenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(p-hydroxyethyl)amino-2-methylaniline, 4-N,N-bis(3-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(0,T-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid, or combinations of two or more thereof.

The p-aminophenol includes, but is not limited to, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid, or combinations of two or more thereof.

The o-aminophenol includes, but is not limited to, 2-aminophenol, 2-amino-5-methylphenol, 2-amino-6-methylphenol and 5-acetamido-2-aminophenol, and the addition salts thereof, or combinations of two or more thereof.

| The bis(phenyl)alkylenediamines that may be mentioned, for example, are N,N′-bis(3-hydroxyethyl)-N,N′-bis(4′-aminophenyl)-1,3-diaminopropano-1, N,N′-bis(3-hydroxyethyl)-N,N′-bis(4′-aminophenyl)ethylenediamine, N,N′-bis(4-aminophenyl)tetramethylenediamine, N,N′-bis(3-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof, or combinations of two or more thereof.

Among the heterocyclic bases that may be mentioned, for example, are pyridine derivatives, pyrimidine derivatives and pyrazole derivatives, or combinations of two or more thereof. Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases can include additional 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-.quadrature.-hydroxyethoxy-3-amino-pyrazolo[1,5-a]pyridine; 2-(4-dimethylpyperazinium-1-yl)-3-amino-pyrazolo[1,5-a]pyridine; and the addition salts thereof, or combinations of two or more thereof.

Nonlimiting examples of direct dyes/pigments includes nitro (hetero) aryl direct dyes such as nitrobenzene and nitropyridine, anthraquinone, nitropyridine, azo, methine, azomethine, xanthene, acridine, azine, carbonyl and tri(hetero)arylmethane direct dyes, and the addition salts thereof; alone or as mixtures. Exemplary direct dyes/pigments also include diazacarbocyanins and isomers thereof, and tetraazacarbocyanins; anthraquinones; naphthoquinone or benzoquinone dyes; indoamine direct dyes; indigoid direct dyes; phthalocyanin and porphyrin direct dyes.

In any embodiment, the coloring agent may include natural dyes including, but not limited to, lawsone, juglone, alizarin, purpurin, carminic acid, kermesic acid, laccaic acid, purpurogallin, anthragallol, protocatechaldehyde, indigo, isatin, curcumin, spinulosin, chlorophylls, chlorophyllines, orceins, haematin, haematoxylin, brazilin, brazileine, safflower dyes (for instance carthamine), flavonoids (with, for example, morin, apigenidin and sandalwood), anthocyans (of the apigeninidin type), carotenoids, tannins, sorghum and cochineal carmine, extracts or decoctions containing these natural dyes, and especially henna-based extracts, or mixtures thereof.

In any embodiment, the composition may include about 0.01 wt % to about 8 wt % of the coloring agent. In any embodiment, the composition may include about 0.1 wt % to about 4 wt % of the coloring agent. In any embodiment, the composition may include about 1 wt % to about 4 wt % of the coloring agent.

In any embodiment, hair-altering composition may further include an antioxidant. For example, the composition may include any of the antioxidants provided in US Patent Appl. Publ. Nos. 2020/0289390, 2020/0360257, 2019/0365625, and 20140338135 and U.S. Pat. Nos. 8,343,238 and 6,818,022, which are each herein incorporated by reference. In any embodiment, the antioxidant may include ascorbic acid, sodium sulfite, sodium metabisulfite, sodium hydrosulfite, tocopherols, a-tocopherol, tocopheryl acetate, panthenol, selenium sulfide, zinc formosulfoxylate, isoascorbic acid, magnesium ascorbyl phosphate, ascorbyl glucoside, cysteine, thiourea, glyceryl monothioglycolate, thioglycerol, 2,5-dihydroxybenzoic acid, erythorbic acid, or combinations of two or more thereof.

In any embodiment, the composition may include about 0.01 wt % to about 5 wt % of the antioxidant. In any embodiment, the composition may include about 0.05 wt % to about 3 wt % of the antioxidant. In any embodiment, the composition may include about 0.1 wt % to about 2 wt % of the antioxidant.

In any embodiment, hair-altering composition may further include one or more additives that alter curl, kink, and/or texture of hair (e.g., relaxation of naturally curly or kinky hair or curling naturally straight hair) (herein referred to as a “curl modifying agent”). In any embodiment, the one or more curl modifying agents may include a hair straightener. In any embodiment, the one or more curl modifying agents may include an alkaline straightener (e.g., guanidine hydroxide, barium hydroxide, lithium hydroxide, sodium hydroxide, and/or potassium hydroxide), thioglycolate, keratin, or combinations of two or more thereof. In any embodiment, the one or more curl modifying agents may include thioglycolate. For example, the composition may include any of the curl modifying agents provided in US Patent Appl. Publ. No. 2013/0306095, U.S. Pat. Nos. 4,898,726 and 7,226,585, and EP 2953610B1, which are each herein incorporated by reference. In any embodiment, the composition may include about 0.1 wt % to about 20 wt % of the curl modifying agent. In any embodiment, the composition may include about 1 wt % to about 15 wt % of the curl modifying agent. In any embodiment, the composition may include about 5 wt % to about 10 wt % of the curl modifying agent. In any embodiment, the composition may include about 3 wt % to about 20 wt % of the curl modifying agent together with the alkaline agent.

In any embodiment, hair-altering composition may further include one or more surface active agents such as surfactants, emulsifiers, and/or solubilizers. For example, the composition may include any of the surface active agents provided in US Patent Appl. Publ. Nos. 2020/0289390, 2020/0360257, 2019/0365625, and 20140338135 and U.S. Pat. Nos. 8,815,225, 8,343,238 and 6,818,022, which are each herein incorporated by reference.

In any embodiment, the surfactant may include an anionic surfactant, nonionic surfactant, amphoteric surfactant, cationic surfactant, or a combination of two or more thereof.

Examples of anionic surfactants include, but are not limited to, sulfate, sulfonate, carboxylate and phosphate type anionic surfactants including C₁₀-C₁₈, C₁₀-C₁₄, and C₁₂-C₁₄ alkyl sulfates and ether sulfates. The sulfate, sulfonate, carboxylate and phosphate type anionic surfactants may include 1 to 50 ethylene oxide group(s) in a molecule (e.g., 1 to 5, 1 to 10, or 1 to 20). Examples of anionic surfactants also include fatty acid amide sulfates, and long-chain mono- and dialkyl phosphates. In any embodiment, the anionic surfactants may include sodium lauryl sulfate and/or sodium laureth sulfate including those with 1 to 50 ethylene oxide group(s) in a molecule (including 1 to 5, 1 to 10, or 1 to 20).

Examples of nonionic surfactants include, but are not limited to, long-chain fatty acid mono- and dialkanolamides such as coconut fatty acid mono- or di-ethanolamide, myristic acid mono- or di-ethanolamide, stearic acid mono- or di-ethanolamide; alkyl polyglucosides having a C₈-C₁₈ alkyl group and one to five glucoside unit(s); sorbitan esters such as polyethylene glycol sorbitan stearic acid, palmitic acid, myristic acid and lauric acid ester; fatty acid polyglycol esters; a polycondensate of ethylene oxide and propylene oxide (commercially available under the trade name of Pluronic®); saturated or unsaturated (poly)oxyethylene alkyl ethers including C₁₀-C₂₂, C₁₆-C₂₀, and C₁₂-C₁₄ alkyl groups and 1 to 100 ethylene oxide group(s) in a molecule (e.g., 1 to 5, 5 to 50, 6 to 40, or 10 to 30).

Examples of amphoteric surfactants include, but are not limited to, various known betaines such as alkyl betaines, fatty acid amido alkyl betaines such as cocamidopropyl betaine, and sulfobetaines like laurylhydroxysulfobetaine, long-chain alkyl amino acids such as cocoaminoacetate, cocoaminopropionate, sodium cocoamphopropionate, and sodium cocoamphoacetate.

Examples of suitable cationic surfactants include, but are not limited to, a mono- or di-long-chain alkyl quaternary ammonium salt of the following general formula: N⁺R¹R²R³R⁴ X⁻. R¹ represents a saturated or unsaturated linear or branched C₅-C₂₂ alkyl group, or a group of R⁵CONH(CH₂)ₙ— or R⁵COO(CH₂)ₙ— (R⁵ represents a saturated or unsaturated linear or branched C₇-C₂₁ alkyl group, and n represents a number of from 1 to 4), R² represents a hydrogen atom, a saturated or unsaturated linear or branched C₁-C₂₂ alkyl group, or a group of the R⁵CONH(CH₂)ₙ— or R⁵COO(CH₂)ₙ—, R³ and R⁴ independently represent a hydrogen atom or a C₁-4 lower alkyl group, and X represents a chloride ion, a bromide ion or a methosulfate ion. Specific examples of the above cationic surfactant include, cetrimonium chloride, steartrimoniun chloride, behentrimonium chloride, dipalmitoyl dimonium chloride, distearyl dimonium chloride, stearamidopropyl trimonium chloride, dioleoylethyl dimonium methosulfate, and dioleoylethylhydroxyethylmonium methosulfate.

In any embodiment, surface active agents may include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, TEA myristate, TEA stearate, magnesium stearate, sodium stearate, polyethyleneoxide stearate (e.g., 1 to 100 ethylene oxide group(s) in a molecule (e.g., 1 to 5, 10 to 80, 20 to 60, or 30 to 50), potassium laurate, potassium ricinoleate, sodium cocoate, sodium tallowate, potassium castorate, sodium oleate, and mixtures thereof. Further exemplary emulsifiers are phosphate esters and the salts thereof such as cetyl phosphate (Amphisol® A), diethanolamine cetyl phosphate (Amphisol®), potassium cetyl phosphate (Amphisol® K), sodium glyceryl oleate phosphate, hydrogenated vegetable glycerides phosphate and mixtures thereof. Furthermore, one or more synthetic polymers may be used. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof. Further exemplary fatty alcohols include cetearyl alcohol (Lanette O, Cognis Coopearation), cetyl alcohol (Lanette 16, Cognis Cooperation), stearyl alcohol (Lanette 18, Cognis Coopearation), Laneth-5 (Polychol 5, Croda Chemicals), furthermore sucrose and glucose derivatives. e.g. sucrose distearate (Crodesta F-10, Croda Chemicals), Methyl glucose isostearate (Isolan IS, Degussa Care Chemicals), furthermore ethoxylated carboxylic acids or polyethyleneglycol esters and polyethyleneglycol ethers, e.g. oleth-20, steareth-2 (Brij 72, Uniqema), steareth-21 (Brij 721, Uniqema), ceteareth-20, ceteareth-25 (Cremophor A25, BASF Cooperation), PEG-40 hydrogenated castor oil (Cremophor RH-40, BASF Cooperation). PEG-7 hydrogenated castor oil (Cremophor WO7, BASF Cooperation), PEG-30 Dipolyhydroxystearate (Arlacel P 135, Unigema), furthermore glyceryl esters and polyglyceryl esters, e.g., C₁₀-C₂₂ glyceryl and polyglyceryl esters, polyglyceryl-3-diisostearate (Hostacerin TGI, Clariant Cooperation), polyglyceryl-2 dipolyhydroxystearate (Dehymuls PGPH, Cognis Cooperation), polyglyceryl-3 methylglucose distearate (Tego Care 450, Degussa Care Chemicals), glyceryl stearate, cetyl phosphate (Amphisol® A), diethanolamine cetyl phosphate (Amphisol®), potassium cetyl phosphate (Amphisol® K), PVP Eicosene copolymer, acrylates/C₁₀₋₃₀-alkyl acrylate crosspolymer, PEG-20 sorbitan isostearate, sorbitan isostearate, and mixtures thereof.

In any embodiment, the one or more surface active agents may include cocamidopropyl betaine, PEG-40 hydrogenated castor oil, sodium lauryl sulfate, sodium laureth sulfate, Oleth-20, glyceryl stearate, PEG-40 stearate, ceteareth-20, or combinations of two or more thereof. In any embodiment, the one or more surface active agents may include cocamidopropyl betaine, PEG-40 hydrogenated castor oil, sodium lauryl sulfate, sodium laureth sulfate, Oleth-20, glyceryl stearate, PEG-40 stearate, ceteareth-20, mineral oil, steareth-20, PEG-4 rapeseedamide, fatty alcohol (e.g., cetearyl alcohol), or combinations of two or more thereof.

In any embodiment, the composition may include about 0.01 wt % to about 20 wt % of the one or more surface active agents. In any embodiment, the composition may include about 0.1 wt % to about 10 wt % of the one or more surface active agents. In any embodiment, the composition may include about 1 wt % to about 8 wt % of the one or more surface active agents.

In any embodiment, hair-altering composition may further include one or more solvents. In any embodiment, the solvent may include water. In any embodiment, the solvent may include an organic solvent. In any embodiment, the organic solvent may include an alcohol. For example, the composition may include any of the solvents provided in US Patent Appl. Publ. Nos. 2020/0289390, 2020/0360257, 2019/0365625, and 20140338135 and U.S. Pat. Nos. 8,815,225, 8,343,238 and 6,818,022, which are each herein incorporated by reference. In any embodiment, the alcohol may include a C₁-C₃₀ alcohol. In any embodiment, the alcohol may include linear saturated C₁₄-C₂₀ alcohol, linear unsaturated C₁₄-C₂₀ alcohol, or a combination thereof. In any embodiment, the alcohol may include cetearyl alcohol, oleyl alcohol, cetyl alcohol, stearyl alcohol, behenyl alcohol, or a combination of two or more thereof.

In any embodiment, the composition may include about 25 wt % to about 75 wt % water. In any embodiment, the composition may include about 30 wt % to about 70 wt % water. In any embodiment, the composition may include about 40 wt % to about 60 wt % water.

In any embodiment, the composition may include about 0.01 wt % to about 10 wt % of the fatty alcohol. In any embodiment, the composition may include about 1 wt % to about 8 wt % of the alcohol.

In any embodiment, the composition may include a skin conditioning agent. For example, the composition may include any of the skin conditioning agents provided in US Patent Appl. Publ. Nos. 2020/0289390, 2020/0360257, 2019/0365625, and 20140338135 and U.S. Pat. Nos. 8,815,225, 8,343,238 and 6,818,022, which are each herein incorporated by reference. In any embodiment, the skin conditioning agent may include silicone materials, amino silicones, C₆-C₃₀ fatty alcohols, polymeric resins, polyol carboxylic acid esters, cationic polymers, cationic surfactants, insoluble oils and oil derived materials, mineral oils and other oils such as glycerin and sorbitol, cationic polymers (e.g., those comprising units of at least one amine group chosen from primary, secondary, tertiary and quaternary amine groups that may either form part of the main polymer chain, or be borne by a side substituent that is directly attached to the main polymer chain), and mixtures thereof.

In any embodiment, the composition may include about 0.01 wt % to about 10 wt % of the conditioning agent. In any embodiment, the composition may include about 0.1 wt % to about 5 wt % of the conditioning agent. In any embodiment, the composition may include about 0.2 wt % to about 4 wt % of the conditioning agent. In any embodiment, the composition may include about 0.2 wt % to about 2 wt % of the conditioning agent.

In any embodiment, the composition may include a fragrance. For example, the composition may include any fragrance provided in US Patent Appl. Publ. Nos. 2020/0289390, 2020/0360257, 2019/0365625, and 20140338135 and U.S. Pat. Nos. 8,815,225, 8,343,238 and 6,818,022, which are each herein incorporated by reference. In any embodiment, the composition may include about 0.001 wt % to about 5 wt % of the fragrance. In any embodiment, the composition may include about 0.005 wt % to about 2 wt % of the fragrance. In any embodiment, the composition may include about 0.01 wt % to about 1 wt % of the fragrance.

In any embodiment, the composition may include a developer. In any embodiment, the developer may include an oxidizing agent. In any embodiment, the oxidizing agent may be present in an amount sufficient to bleach the melanin pigment in the hair and/or oxidize dye/pigment precursors. In any embodiment, the oxidizing agent may include a peroxide including, but not limited to, hydrogen peroxide; inorganic alkali metal peroxides (e.g. sodium periodate and sodium peroxide); organic peroxides (e.g. urea peroxide, melamine peroxide); inorganic perhydrate salt bleaching compounds (e.g. alkali metal salts of perborates, percarbonates, perphosphates, persilicates, and persulphates, in some embodiments, sodium salts thereof), which may be incorporated as monohydrates, tetrahydrates, etc.; alkali metal bromates; enzymes; and mixtures thereof. In any embodiment, the oxidizing agent may include hydrogen peroxide.

In any embodiment, the developer may include about 5 wt % to about 80 wt % of the oxidizing agent. In any embodiment, the developer may include about 5 wt % to about 50 wt % of the oxidizing agent. In any embodiment, the developer may include about 10 wt % to about 40 wt % of the oxidizing agent. In any embodiment, the developer may include about 25 wt % to about 35 wt % of the oxidizing agent. In any embodiment, the developer may include about 15 wt % to about 25 wt % of the oxidizing agent. In any embodiment, the developer may include about 5 wt % to about 15 wt % of the oxidizing agent.

In any embodiment, the composition may include about 1 wt % to about 20 wt % of the oxidizing agent. In any embodiment, the composition may include about 1 wt % to about 15 wt % of the oxidizing agent. In any embodiment, the composition may include about 3 wt % to about 12 wt % of the oxidizing agent. In any embodiment, the composition may include about 6 wt % to about 10 wt % of the oxidizing agent.

In any embodiment, the composition may include a chelating agent. In any embodiment, the chelating may include a compound or ligand that can bind to a metal ion, usually through more than one ligand atom, to form a chelate. A chelate is usually a type of coordination compound in which a central metal ion, such as Co²⁺, Ni²⁺, Cu²⁺, Ca²⁺ or Zn²⁺, is attached by coordinate links to two or more nonmetal atoms, i.e., ligands, in the same molecule. In any embodiment, the chelating agent may include, but is not limited to, ethylene-diaminetetraacetic acid (EDTA), nitrilotriacetic acid, ethyleneglycol-bis(3-amino-ethyl ether)-N,N-tetraacetic acid, and salts of any of the foregoing.

In any embodiment, the composition may include about 0.001 wt % to about 5 wt % of the chelating agent. In any embodiment, the composition may include about 0.01 wt % to about 2 wt % of the chelating agent. In any embodiment, the composition may include about 0.1 wt % to about 1 wt % of the chelating agent. In any embodiment, the composition may include about 0.1 wt % to about 0.5 wt % of the chelating agent.

In any embodiment, the composition may have a pH at or above 7. In any embodiment, the composition may have a pH of about 7.5 to about 13. In any embodiment, the composition may have a pH of about 8 to about 12.5. In any embodiment, the composition may have a pH of about 9 to about 12. In any embodiment, the composition may have a pH of about 10 to about 10.5.

In any embodiment, the composition may include DL-2AP as well as a fatty alcohol, a fatty acid, a cationic surfactant, a quaternary polymer, or combinations of two or more thereof including any of those disclosed herein. In any embodiment, the composition may include DL-2AP as well as a fatty alcohol, a fatty acid, a cationic surfactant, a quaternary polymer, peroxygenated salts (e.g., sodium, potassium, and/or ammonium persulfates), a mineral (e.g., magnesium carbonate (optionally in hydrate form)), a swelling agent (e.g., carboxymethyl cellulose and/or gum), or combinations of two or more thereof including any of those disclosed herein. In any embodiment, the composition may include DL-2AP as well as water, lauric acid, cetearyl alcohol, polyoxyethylene alkyl (12-14) ether (3 EO), polyoxyethylene lauryl ether (12 EO), polyoxyethylene oleyl ether, glycol distearate, titanium dioxide, dimethicone/dimethicone copolymer, propylene glycol, hexadimethrine chloride, polychloride dimethyl pyrrolidinium solution, dihydrosphingosine oleate, silica silylate, EDTA, carbomer, fragrance, or combinations of two or more thereof. In any embodiment, the composition may in the form of a cream.

In any embodiment, the hair-altering composition may optionally further include one or more preservatives. Such preservatives are well known to those skilled in the art. For example, the preservatives may include, but are not limited to, aldehydes (e.g., formaldehyde, DMDM hydantoin, imadozolidinyl urea, diazolidinyl urea; as safeguard against bacteria and some fungi); glycol ethers (e.g., phenoxyethanol and caprylyl glycol; as safeguard against some bacteria); isothiazolinones (e.g., methylisothiazolinone; as safeguard against bacteria and fungi); organic acids (e.g., benzoic acid, sorbic acid, levulinic acid, anisic acid; as safeguard against fungi and some bacteria); parabens (e.g., methylparaben, ethylparaben, propylparaben, butylparaben, isobutylparaben; as safeguard against fungi and some bacteria), or any combination thereof.

In any embodiment, the composition may include peroxygenated salts (e.g., sodium, potassium, and/or ammonium persulfates), a mineral (e.g., magnesium carbonate (optionally in hydrate form)), a swelling agent (e.g., carboxymethyl cellulose and/or gum), or combinations of two or more thereof including any of those disclosed herein. In any embodiment, the composition may include potassium persulfate, sodium persulfate, ammonium chloride, calcium stearate, hydrated silica, liquid paraffin, titanium dioxide, sodium lauryl sulfate, anhydride sodium metasilicate, EDTA, guar gum, or combinations of two or more thereof including any of those disclosed herein. In any embodiment, the composition may in the form of a powder. In any embodiment, the developer may include water, hydrogen peroxide, a fatty alcohol a surfactant, phosphoric acid, a chelating agent, or combinations of two or more thereof including any of those disclosed herein. In any embodiment, the composition may also include water, cetearyl alcohol, behentrimonium chloride, amodimethicone, cetyl alcohol, C₁₂-C₁₅ alkyl benzoate, guarhydroxypropyl trimonium chloride, coconut oil, hydroxyethylcellulose, sodium hydroxide, trideceth-6, chlorhexidine digluconate, fumaric acid, isopropanol, citric acid, cetrimonium chloride, or combinations of two or more thereof.

In any embodiment, the composition may be in the form of a liquid, gel, cream, paste, emulsion, suspension, or foam. In any embodiment, the composition may be in the form of a gel. In any embodiment, the composition may be in the form of a cream.

In any embodiment, hair treated with the composition may exhibit a hair color altering ΔE of less than about 2.5 compared to a control composition having ammonia substituted for the DL-2AP. In any embodiment, the control composition has about 1:1 mole substitution of the DL-2AP for ammonia. In any embodiment, the hair treated with the composition may exhibit a hair color altering ΔE of less than or equal to about 2 compared to the control composition. In any embodiment, the hair treated with the composition may exhibit a hair color altering ΔE of less than or equal to about 1.95 compared to the control composition. In any embodiment, the hair treated with the composition may exhibit a hair color altering ΔE of less than or equal to about 1.9 compared to the control composition. In any embodiment, the hair treated with the composition may exhibit a hair color altering ΔE of less than or equal to about 1.75 compared to the control composition. In any embodiment, the hair treated with the composition may exhibit a hair color altering ΔE of less than or equal to about 1.6 compared to the control composition. In any embodiment, the hair treated with the composition may exhibit a hair color altering ΔE of less than or equal to about 1.5 compared to the control composition. In any embodiment, the hair treated with the composition may exhibit a hair color altering ΔE of less than or equal to about 1.25 compared to the control composition. In any embodiment, the hair treated with the composition may exhibit a hair color altering ΔE of less than or equal to about 1 compared to the control composition. In any embodiment, the hair treated with the composition may exhibit a hair color altering ΔE of less than or equal to about 0.75 compared to the control composition.

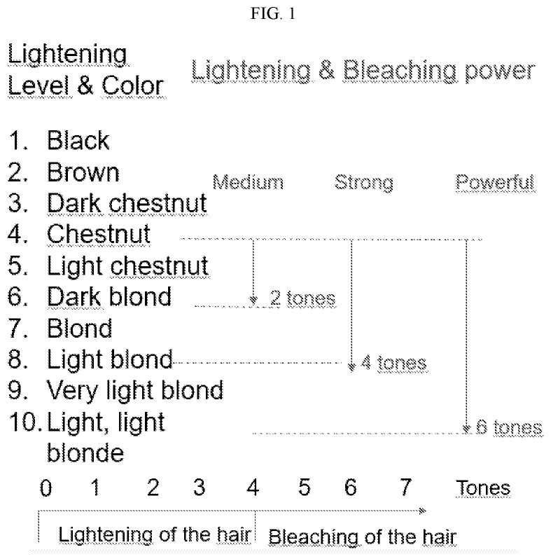

It is well known in the art that color lifting, sometimes referred to as bleaching, is commonly needed to achieve a desired color alteration. In particular, color lifting is needed when a lighter tone is desired. It also aids in achieving similar color altering of gray and pigmented hair. Color lifting is typically referred to in terms of tone change (FIG. 1). In any embodiment, the hair treated with the composition may exhibit a color tone change of at least about 2. In any embodiment, the hair treated with the composition may exhibit a color tone change of at least about 2.5. In any embodiment, the hair treated with the composition may exhibit a color tone change of at least about 3. In any embodiment, the hair treated with the composition may exhibit a color tone change of at least about 3.5. In any embodiment, the hair treated with the composition may exhibit a color tone change of at least about 4.

In any embodiment, the hair treated with the composition may exhibit a cysteic acid difference of less than about 40% compared to the control composition. In any embodiment, the hair treated with the composition may exhibit a cysteic acid difference of less than about 35% compared to the control composition. In any embodiment, the hair treated with the composition may exhibit a cysteic acid difference of less than about 30% compared to the control composition. In any embodiment, the hair treated with the composition may exhibit a cysteic acid difference of less than about 25% compared to the control composition. In any embodiment, the hair treated with the composition may exhibit a cysteic acid difference of less than about 20% compared to the control composition. In any embodiment, the ΔE and/or the cysteic acid is measured after one color treatment cycles. In any embodiment, the ΔE and/or the cysteic acid is measured after two or more color treatment cycles. In any embodiment, the ΔE and/or the cysteic acid is measured after three or more color treatment cycles. In any embodiment, the ΔE and/or the cysteic acid is measured after five color treatment cycles.

In another aspect, the present application provides kits including the hair-altering composition. The kit may include a first composition that includes the DL-2AP. In any embodiment, the kit may include the first composition and a second composition that includes the developer. Following combination of the first composition and the second composition, the hair-altering compositions disclosed herein are achieved. In any embodiment, the developer may include the oxidizing agent(s) disclosed herein. In any embodiment, the first composition may include the coloring agent(s) disclosed herein. In any embodiment, the first composition may include the curl modifying agent(s) disclosed herein. In any embodiment, the first composition may include the pH adjusting agent(s) disclosed herein. In any embodiment, first composition and/or the second composition may include any of the other components disclosed herein including the alkanolamine(s) different from 2-aminopropanol, ammonia, arginine, or a combination thereof, the antioxidant(s), the curl modifying agent(s), the pH adjusting agent(s), the surface active agent(s), the solvent(s), the skin conditioning agent(s), the fragrance(s), the chelating agent(s), or combination of two or more thereof. In any embodiment, first composition may include any of the other components disclosed herein including the alkanolamine(s) different from 2-aminopropanol, ammonia, arginine, or a combination thereof, the antioxidant(s), the curl modifying agent(s), the pH adjusting agent(s), the surface active agent(s), the solvent(s), the skin conditioning agent(s), the fragrance(s), the chelating agent(s), or combination of two or more thereof. The kit may further include a shampoo and/or a conditioner for use in rinsing the hair after altering. In any embodiment, the kit may further include a third composition. In any embodiment, the third composition may include peroxygenated salts (e.g., sodium, potassium, and/or ammonium persulfates), a mineral (e.g., magnesium carbonate (optionally in hydrate form)), a swelling agent (e.g., carboxymethyl cellulose and/or gum), or combinations of two or more thereof including any of those disclosed herein. In any embodiment, the first composition may in the form of a cream. In any embodiment, the second composition may in the form of a liquid. In any embodiment, the third composition may in the form of a powder.

The present application also provides a method for altering hair including applying the composition disclosed herein on a hair; and rinsing the composition off of the hair with water to provide the altered hair. In any embodiment, the rinsing may occur at least about 10 minutes after the applying. In any embodiment, the rinsing may occur at least about 20 minutes after the applying. In any embodiment, the rinsing may occur at least about 30 minutes after the applying. In any embodiment, the method may further include applying heat to the hair after applying the composition. In any embodiment, the altered hair may be color altered. In any embodiment, the color-altered hair may exhibit ΔE and/or cysteic acid difference as disclosed herein. In any embodiment, the altered hair may be curl, kink, or hair texture altered. In any embodiment, the hair-altering composition is a keratin altering composition and/or disulfide bond breaking composition. In any embodiment, the method may further include applying heat to the hair after applying the composition. In any embodiment, the heat may be applied by a flat iron. In any embodiment, the altered hair may be color altered and curl, kink, or hair texture altered.

The examples herein are provided to illustrate advantages of the present technology and to further assist a person of ordinary skill in the art with preparing or using the compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

### Example 1. Methods and Materials

Alkalizer Systems. Ammonia, as ammonium hydroxide, 28-30% active material, was obtained from Aldrich. Monoethanolamine (MEA), 99% active material, was obtained from Dow Chemical Company. DMAMP and DL-2AP samples were obtained from ANGUS Chemical Company. DMAMP was included in testing, except for color lift testing. Conditioning cream base formulations were prepared in laboratory, and alkalizers were added in the desired amount (w/w %) prior to testing and mixed well. Conditioner base master batch was prepared with ˜10% gap to allow for addition of the alkalizing agents.

Type of Hair Used in Testing: 1. Virgin brown hair for color lifting application; 2. 95% gray hair for blond color; 3. Twice bleached hair for dark brown and red colors; 4. 75% gray hair for gray coverage application; and 5. 100% natural white hair for medium brown color

Hair tress preparation. Hair tresses were purchased and washed thoroughly before usage. Suave Clarifying Shampoo solution at 2.5% w/v was prepared by mixing 25 g shampoo in 1000 mL warm tap water. Five hair tresses were clipped together and soaked in the shampoo solution for 10 minutes. Tresses were rinsed in warm water and allowed to air dry.

Application Protocol. The color lift formula was completed by mixing the base with the desired alkalizing agent until the target pH was obtained. This fully formulated cream was then activated by the addition of peroxide in a 1:1.5 ratio w/w. 30-volume peroxide was used for the lifting test. Eight grams of the resultant cream color formula was applied to hair tresses and spread evenly through the hair. It was allowed to stand and react for 50 minutes at room temperature. Tresses were rinsed with tap water and allowed to air dry.

A similar protocol was followed for color deposition tests, with 20-volume peroxide for the dark brown color and 30-volume peroxide for blond and red color formulations.

Colorfastness. Retention of color in hair is measured using repeated washing of hair. Tresses were wet under running tap water, 0.2 g Suave Clarifying Shampoo was applied and rubbed throughout for 30 seconds. Tresses were then rinsed with warm water for 90 seconds and dried in a convection oven at 40° C. until completely dried. This protocol was repeated for 15 cycles, and color readings were recorded every 5 cycles.

Hair Damage. The hair damage was measured by monitoring increase in cysteic acid using FTIR-ATR technique. For color lift test, virgin brown hair was used as the undamaged control, while twice bleached hair was the negative control. Measurements were conducted after one and five color treatments, with 5 washes in between color treatments. Scanning electron micrographs were taken to visually assess hair damage.

### Example 2. Color Lift Compositions

All components in Phase A were mixed and heated to 75° C. to provide Phase A. All components in Phase B were mixed and heated to 75° C. to provide Phase B. Under stirring Phase A was poured into Phase B and stirring was continued for 5 minutes. Heat was removed and the mixture was stirred for another 30 minutes prior to adding Phase C to provide the alkaline composition. The alkaline composition was mixed with a 30 vol % hydrogen peroxide composition at a ratio volume of 1:1.5 (alkaline composition:hydrogen peroxide composition) to provide the color lift compositions (Table 1). Each color lift composition was applied to virgin hair samples. After 50 minutes at room temperature, the color lift compositions were washed from the hair samples with tap water until clean and the hair was air dried to provide the color-altered hair samples. As discussed above, the calculation of ΔE is useful in industry for detecting small differences in color. A ΔE result with a difference less than 2 compared the ammonia treated hair is considered to be equal performance (i.e., not visible to the naked eye). Using BYK Gardner Spectro-Guide Sphere, the ΔE of each color-altered hair sample was measured and the difference compared to the ammonia treated hair sample reported (Table 2). A second set of virgin hair samples were treated with the color lift compositions following the same method 5-times (total of 5 cycles). The ΔE of each color-altered hair sample was measured after one cycle and after five cycles (Table 3).

TABLE 1
Color Lift Compositions

| | | | |
|---|---|---|---|
| | Ammonia | DL-2AP | MEA |
| | Compo- | Compo- | Compo- |
| | sition | sition | sition |
| Component | (wt %) | (wt %) | (wt %) |

Phase A

| | | | |
|---|---|---|---|
| Water | q.s. | q.s. | q.s. |
| Tetrasodium EDTA | 0.3 | 0.3 | 0.3 |
| Cocamidopropyl Betaine | 2.00 | 2.00 | 2.00 |
| PEG-40 Hydrogenated | 2.0 | 2.0 | 2.0 |
| Castor Oil (Lipocol | | | |
| HCO-40) | | | |
| Propylene Glycol | 2.50 | 2.50 | 2.50 |

Phase B

| | | | |
|---|---|---|---|
| Cetearyl alcohol | 15.75 | 15.75 | 15.75 |
| (Vegarol 1618) | | | |
| Oleth-20 (Lipocol O-20) | 3.50 | 3.50 | 3.50 |
| Simmondsia Chinensis | 2.00 | 2.00 | 2.00 |
| (jojoba) butter (Isojojoba | | | |
| 50) | | | |
| Ceteareth-20 | 10.00 | 10.00 | 10.00 |

Phase C

| | | | |
|---|---|---|---|
| Ammonia (28-30%) | 5-11 | — | — |
| DL-2AP | — | 5-11 | — |
| MEA | | — | 5-11 |
| DMAMP | — | — | — |
| Total | 100 | 100 | 100 |

Color Lift Composition Properties

| | | | |
|---|---|---|---|
| pH | 9.7-10 (for | 9.8-10.1 @ | 9.8-10.2 @ |
| | 5% and 11%) | 5% to 11% | 5% to 11% |
| physical | Cream | Cream | Cream |

TABLE 2
Color Alteration of Hair based ΔE Measurement (1 Cycle)

| | Concentration | | | | | | | |
|---|---|---|---|---|---|---|---|---|
| | 5% | | 7% | | 9% | | 11% | |
| | ΔE | | ΔE | | ΔE | | ΔE | |
| | Virgin | ΔE | Virgin | ΔE | Virgin | ΔE | Virgin | ΔE |
| System | Hair | ammonia | Hair | ammonia | Hair | ammonia | Hair | ammonia |
| Ammonia | 11.2 | — | 12.27 | — | 12.39 | — | 13.64 | — |
| MEA | 11.1 | 0.41 | 11.64 | 0.93 | 11.98 | 0.48 | 11.81 | 1.89 |
| DL-2AP | 8.93 | 2.33 | 11.26 | 1.26 | 11.78 | 0.80 | 12.10 | 1.57 |

TABLE 3
Color Alteration of Hair based ΔE Measurement (1 and 5 Cycles)

| | Concentration | | | | | | | |
|---|---|---|---|---|---|---|---|---|
| | 7% (1 Cycle) | | 11% (1 Cycle) | | 7% (5 Cycles) | | 11% (5 Cycles) | |
| | ΔE | | ΔE | | ΔE | | ΔE | |
| | Virgin | ΔE | Virgin | ΔE | Virgin | ΔE | Virgin | ΔE |
| System | Hair | ammonia | Hair | ammonia | Hair | ammonia | Hair | ammonia |
| Ammonia | 11.79 | — | 13.11 | — | 27.82 | — | 28.34 | — |
| MEA | 10.96 | 0.84 | 11.86 | 1.34 | 27.26 | 1.43 | 27.64 | 1.71 |
| DL-2AP | 10.45 | 1.38 | 11.52 | 1.71 | 25.97 | 2.14 | 26.27 | 2.49 |

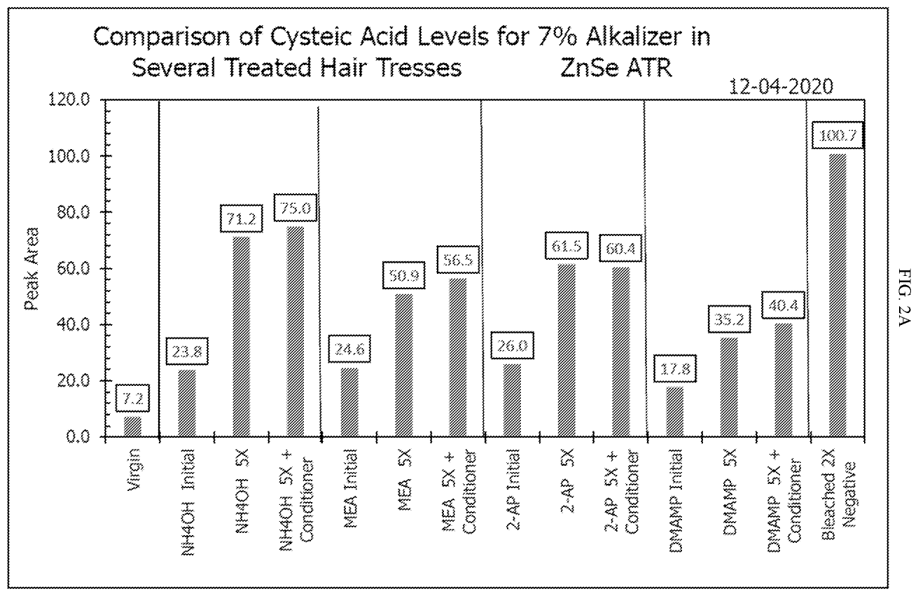

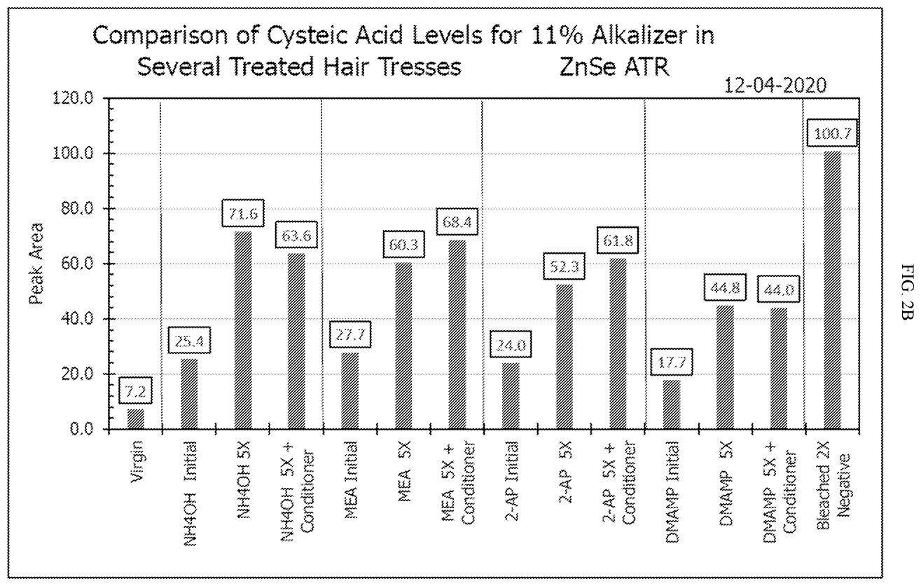

The cysteic acid content in the virgin hair and color altered hair samples (after 1 and 5 treatment cycles) having 7 wt % or 11 wt % of the alkalizing agent were determined using FTIR-ATR by measuring disulfide bonds (—S—S—) via reduction and oxidation (measured 5-times with averages reported) (FIGS. 2A and 2B). The presence and intensity of the S03 band at 1040 cm⁻¹ indicates the disulfide bonds that had been cleaved and then oxidized to residues of cysteic acid due to treatment of the hair with an oxidizing agent (e.g., hydrogen peroxide), UV radiation, or chemical straightening procedures (see V. Signoir, et al., Int. J Cosmet. Sci. 19, 1-13 (1997). A higher cysteic acid content indicates higher damage to hair.

### Example 3. Color Deposition

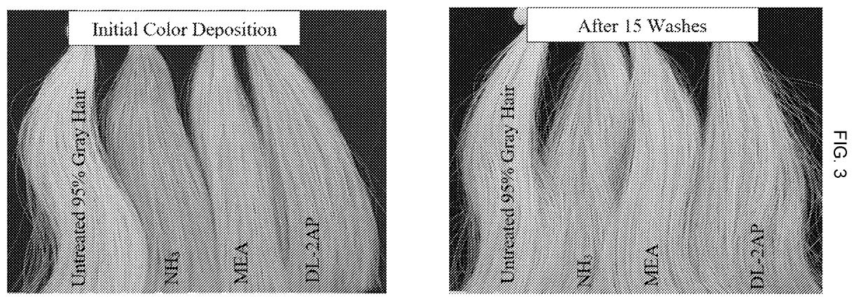

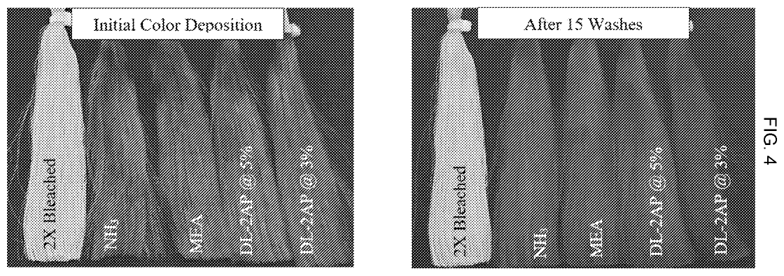

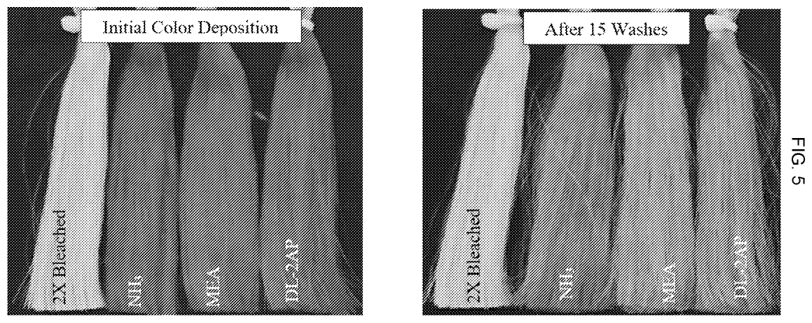

Performance of DL-2AP on color development was tested for three color families, namely blond, dark brown and red. The alkalizer performance was compared with industry standards of ammonia and MWA. For the 3 color families, the best starting hair were chosen to be able to differentiate resulting color. Blond hair color was evaluated on 95% virgin gray hair. Dark brown and red colors were evaluated on twice bleached hair. The permanent color development and fastness of that color are reported in FIGS. 3-5.

### Example 3.1. Blond Color Compositions

The blond color alkaline compositions containing, ammonia, MEA, or DL-2AP (Table 4) were prepared according to the procedure:
1. Prepare A: Combine ingredients under stirring and heat until temperature reaches 75° C.
2. Combine B ingredients into a separate mixture and heat to 75° C.
3. Emulsify B into A using a homogenizer at 4000 rpm for 3 min. Cool mixture down to 40° C. under stirring using a paddle blade at 200 rpm.
4. Calculate water loss and adjust accordingly.
5. Add C and D mix for 10 min.

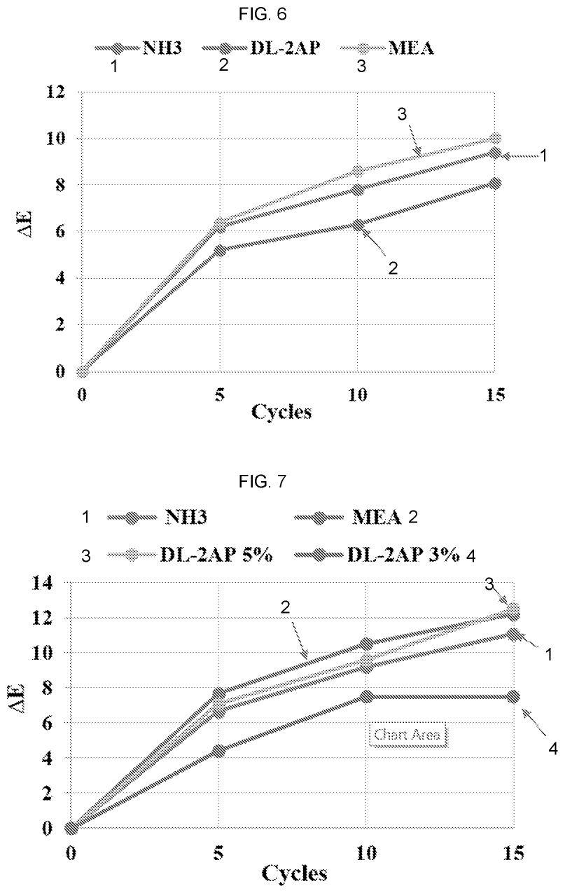

Hair samples (twice bleached) were treated with the blond compositions. The ΔE of each color-altered hair sample was measured and the difference of the DL-2AP treated hair sample was compared to the MVWA or ammonia treated hair sample (Table 5). FIG. 6 presents the color fastness differences. DL-2AP shows comparable color fastness as NI-1 and MEA.

TABLE 4
Blond Color Compositions

| | | | |
|---|---|---|---|
| | Ammonia | DL-2AP | MEA |
| | Compo- | Compo- | Compo- |
| | sition | sition | sition |
| Component | (wt %) | (wt %) | (wt %) |

Blond Color Alkaline Compositions
Phase A1

| | | | |
|---|---|---|---|
| Water | 49.30 | 49.30 | 49.30 |
| Tetrasodium EDTA | 0.30 | 0.30 | 0.30 |
| Cocamidopropyl Betaine | 2.00 | 2.00 | 2.00 |
| PEG-40 Hydrogenated | 2.00 | 2.00 | 2.00 |
| Castor Oil (Lipocol HCO- | | | |
| 40) | | | |
| Sodium metabisulphite | 1.00 | 1.00 | 1.00 |
| Ascorbic acid | 0.30 | 0.30 | 0.30 |

Phase A2

| | | | |
|---|---|---|---|
| p-aminophenol | 0.327 | 0.327 | 0.327 |
| Resorcinol | 0.220 | 0.220 | 0.220 |
| 4-Amino-2- | 0.037 | 0.037 | 0.037 |
| hydroxytoluene | | | |
| m-aminophenol | 0.076 | 0.076 | 0.076 |

Phase B

| | | | |
|---|---|---|---|
| Cetearyl alcohol (Vegarol | 16.50 | 16.50 | 16.50 |
| 1618) | | | |
| Oleth-20 (Lipocol O-20) | 3.50 | 3.50 | 3.50 |
| Oleyl alcohol (Lipocol | 3.00 | 3.00 | 3.00 |
| O/95) | | | |
| Simmondsia Chinensis | 2.00 | 2.00 | 2.00 |
| (jojoba) butter (Isojojoba | | | |
| 50) | | | |
| Cetearyl alcohol/glyceryl | 10.00 | 10.00 | 10.00 |
| stearate/PEG-40 | | | |
| stearate/ceteareth-20 | | | |
| (Lipomulse Luxe) | | | |

Phase C

| | | | |
|---|---|---|---|
| Preservative (customer | q.s. | q.s. | q.s. |
| dependent) | | | |

Phase D

| | | | |
|---|---|---|---|
| Ammonia (28-30%) | 9 | — | — |
| DL-2AP | — | 9 | — |
| MEA | — | — | 9 |

Blond Color Composition Properties

| | | | |
|---|---|---|---|
| pH | 9.56 | 9.67 | 9.89 |
| physical | Cream | Cream | Cream |

TABLE 5
Color Alteration of Hair with Blond Color Compositions (1 Cycle)

| | ΔE | Color Parameters | | |
|---|---|---|---|---|
| | 1 Treatment | L | a | b |
| NH₃ | 17.4 | 54.4 | 14.5 | 21.8 |
| MEA | 16.7 | 55.6 | 15.0 | 20.8 |
| DL-2AP | 14.6 | 56.9 | 13.5 | 20.0 |

### Example 3.2. Dark Brown Color Compositions

The dark brown color compositions containing MEA, ammonia, or DL-2AP (Table 6) were prepared according to the same procedure as specified for the blond color compositions of Example 3.1.

Hair samples (twice bleached) were treated with the dark brown color compositions. The ΔE of each color-altered hair sample was measured and the difference of the DL-2AP treated hair sample was compared to the MEA or ammonia treated hair sample (Table 7). DL-2AP presented excellent color deposition, with noticeable differences in red and yellow coordinates compared to MEA or NH₃. Wash fastness in FIG. 7 shows that DL-2AP at reduced level displays improved color fastness vs. controls after multiple shampoo washes.

TABLE 6
Dark Brown Color Compositions

| | | | | |
|---|---|---|---|---|
| | Ammonia | DL-2AP | DL-2AP | MEA |
| | Compo- | Compo- | Compo- | Compo- |
| | sition | sition | sition | sition |
| Component | (wt %) | (wt %) | (wt %) | (wt %) |

Dark Brown Color Alkaline Compositions
Phase A1

| | | | | |
|---|---|---|---|---|
| Water | q.s. | q.s. | q.s. | q.s. |
| Tetrasodium EDTA | 0.30 | 0.30 | 0.30 | 0.30 |
| Cocamidopropyl Betaine | 2.00 | 2.00 | 2.00 | 2.00 |
| PEG-40 Hydrogenated | 2.00 | 2.00 | 2.00 | 2.00 |
| Castor Oil (Lipocol HCO- | | | | |
| 40) | | | | |
| Sodium metabisulphite | 1.00 | 1.00 | 1.00 | 1.00 |
| Ascorbic acid | 0.30 | 0.30 | 0.30 | 0.30 |

Phase A2

| | | | | |
|---|---|---|---|---|
| p-aminophenol | 1.09 | 1.09 | 1.09 | 1.09 |
| Resorcinol | 0.11 | 0.11 | 0.11 | 0.11 |
| 4-Amino-2-hydroxytoluene | 0.246 | 0.246 | 0.246 | 0.246 |
| m-aminophenol | 1.090 | 1.090 | 1.090 | 1.090 |
| N,N-Bis-(2-hydroxyethyl)- | 0.624 | 0.624 | 0.624 | 0.624 |
| p-phenylenediamine Sulfate | | | | |
| 2-Methoxymethyl-p- | 0.304 | 0.304 | 0.304 | 0.304 |
| Phenylenediamine | | | | |
| Hydroxyethyl-3,4- | 0.218 | 0.218 | 0.218 | 0.218 |
| Methylenedioxyaniline | | | | |
| Hydrochloride | | | | |

Phase B

| | | | | |
|---|---|---|---|---|
| Cetearyl alcohol (Vegarol | 15.0 | 15.0 | 15.0 | 15.0 |
| 1618) | | | | |
| Oleth-20 (Lipocol O-20) | 3.50 | 3.50 | 3.50 | 3.50 |
| Oleyl alcohol (Lipocol | 3.00 | 3.00 | 3.00 | 3.00 |
| O/95) | | | | |
| Simmondsia Chinensis | 2.00 | 2.00 | 2.00 | 2.00 |
| (jojoba) butter (Isojojoba | | | | |
| 50) | | | | |
| Cetearyl alcohol/glyceryl | 10.00 | 10.00 | 10.00 | 10.00 |
| stearate/PEG-40 | | | | |
| stearate/ceteareth-20 | | | | |
| (Lipomulse Luxe) | | | | |

Phase C

| | | | | |
|---|---|---|---|---|
| Preservative (Customer | q.s. | q.s. | q.s. | q.s. |
| dependent) | | | | |

Phase D

| | | | | |
|---|---|---|---|---|
| Ammonia (28-30%) | 5 | — | — | — |
| DL-2AP | — | 5 | — | — |
| | — | — | 3 | — |
| MEA | — | — | — | 5 |

Dark Brown Color Composition Properties

| | | | | |
|---|---|---|---|---|
| pH | 9.46 | 9.5 | 9.27 | 9.7 |
| physical | Cream | Cream | Cream | Cream |

TABLE 7
Color Alteration of Hair with Dark
Brown Color Compositions (1 Cycle)

| | ΔE | Color Parameters | | |
|---|---|---|---|---|
| | 1 Treatment | L | a | b |
| NH₃ | 48.9 | 19.5 | 3.9 | 2.0 |
| MEA | 51.0 | 17.9 | 2.5 | 0.2 |
| DL-2AP 5% | 50.33 | 18.7 | 3.3 | 0.1 |
| DL-2AP 3% | 50.80 | 18.3 | 3.0 | −0.1 |

### Example 3.3. Red Color Compositions

The red color compositions containing MEA, ammonia, or DL-2AP (Table 8) were prepared according to the same procedure as specified for the blond color compositions of Example 3.1.

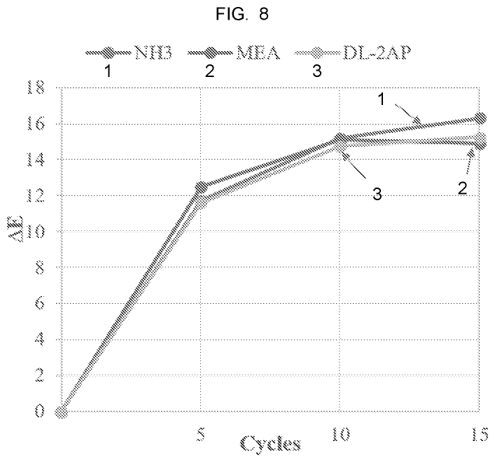

Hair samples (twice bleached) were treated with the red color compositions. The ΔE of each color-altered hair sample was measured and the difference of the DL-2AP treated hair sample was compared to the MEA or ammonia treated hair sample (Table 9). FIG. 8 presents the color fastness differences. DL-2AP shows comparable color fastness as NH₃ and MEA.

TABLE 8
Red Color Compositions

| | | | |
|---|---|---|---|
| | Ammonia | MEA | DL-2AP |
| | Compo- | Compo- | Compo- |
| | sition | sition | sition |
| Component | (wt %) | (wt %) | (wt %) |

Red Color Alkaline Compositions

| | | | |
|---|---|---|---|
| Water (Aqua) | q.s. | q.s. | q.s. |
| Disodium | 0.30 | 0.30 | 0.30 |
| Ethylenediaminetetraacetate | | | |
| Cocamidopropyl Betaine | 2.00 | 2.00 | 2.00 |
| PEG-40 Hydrogenated Castor | 2.00 | 2.00 | 2.00 |
| Oil | | | |
| Sodium Metabisulfite | 1.00 | 1.00 | 1.00 |
| Ascorbic Acid | 0.30 | 0.30 | 0.30 |

Phase A2

| | | | |
|---|---|---|---|
| 2-Methoxymethyl-p- | 0.152 | 0.152 | 0.152 |
| Phenylenediamine | | | |
| 1-Hydroxyethyl-4,5- | 1.440 | 1.440 | 1.440 |
| Diaminopyrazole Sulfate | | | |
| 4-Amino-2-hydroxytoluene | 0.123 | 0.123 | 0.123 |
| m-aminophenol | 0.545 | 0.545 | 0.545 |
| Resorcinol | 0.055 | 0.055 | 0.055 |
| Hydroxyethyl-3,4- | 0.109 | 0.109 | 0.109 |
| Methylenedioxyaniline | | | |
| Hydrochloride | | | |

Phase B

| | | | |
|---|---|---|---|
| Cetearyl Alcohol | 15.50 | 15.50 | 15.50 |
| Oleth-20 | 3.50 | 3.50 | 3.50 |
| Oleyl Alcohol | 3.00 | 3.00 | 3.00 |
| Simondsia Chinensis (Jojoba) | 2.00 | 2.00 | 2.00 |
| Butter | | | |
| Cetearyl Alcohol (and) | 10.00 | 10.00 | 10.00 |
| Glyceryl Stearate (and) PEG- | | | |
| 40 Stearate (and) Ceteareth-20 | | | |

Phase C

| | | | |
|---|---|---|---|
| Customer dependent | q.s. | q.s. | q.s. |

Phase D

| | | | |
|---|---|---|---|
| Aminopropanol | — | — | 5.00 |
| MEA | — | 5.00 | — |
| Ammonia (28-30%) | 5.00 | — | — |

Red Color Composition Properties

| | | | |
|---|---|---|---|
| pH | 9.38 | 9.7 | 9.55 |
| physical | Cream | Cream | Cream |

TABLE 9
Color Alteration of Hair with Red Color Compositions

| | ΔE | Color Parameters | | |
|---|---|---|---|---|
| | 1 Treatment | L | a | b |
| NH₃ | 44.8 | 23.3 | 18.4 | 8.7 |
| MEA | 44.2 | 24.4 | 20.6 | 9.7 |
| DL-2AP | 44.3 | 24.0 | 19.2 | 9.2 |

### Example 4. Straightening Compositions

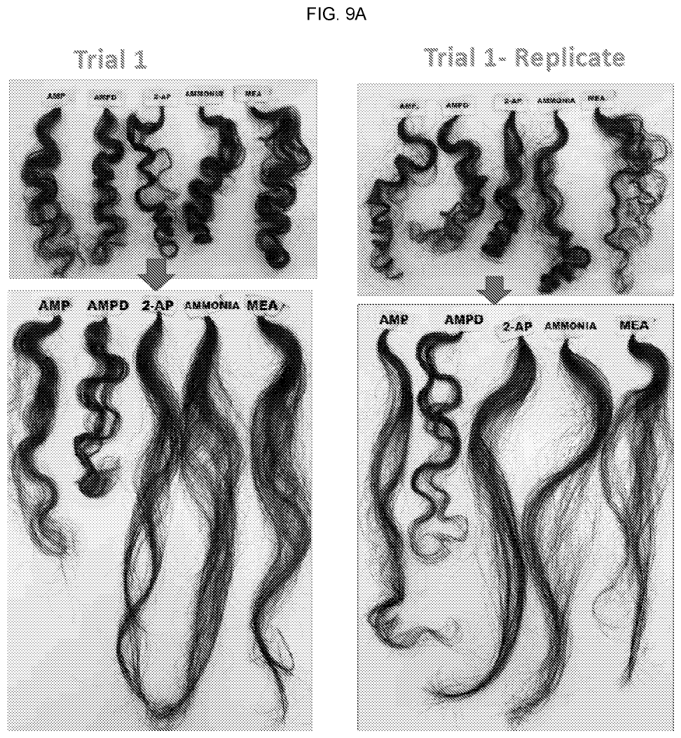

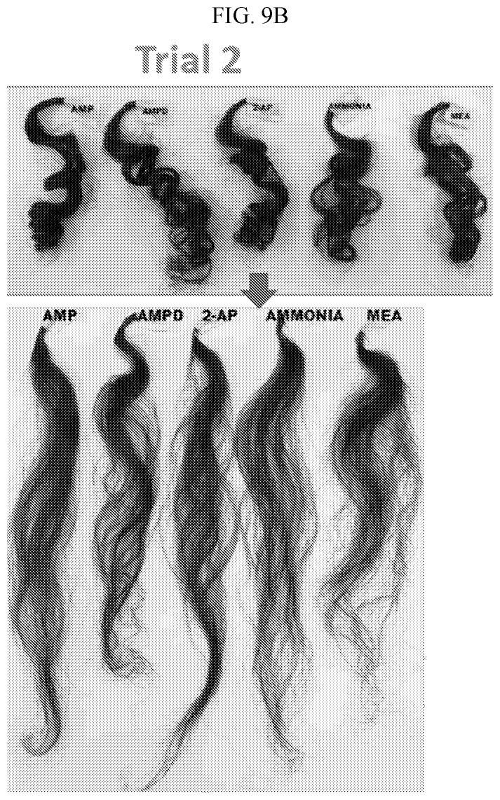

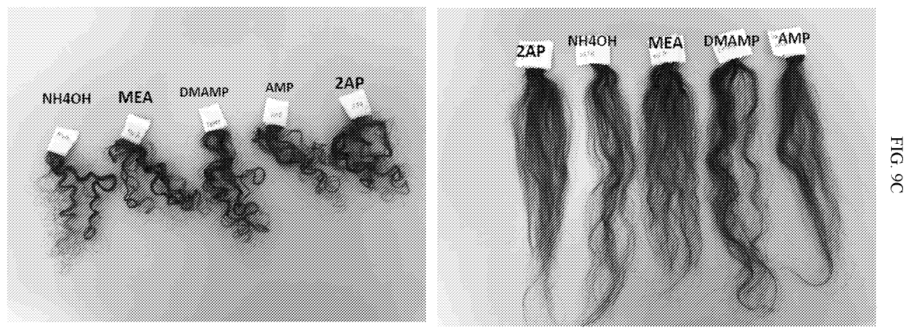

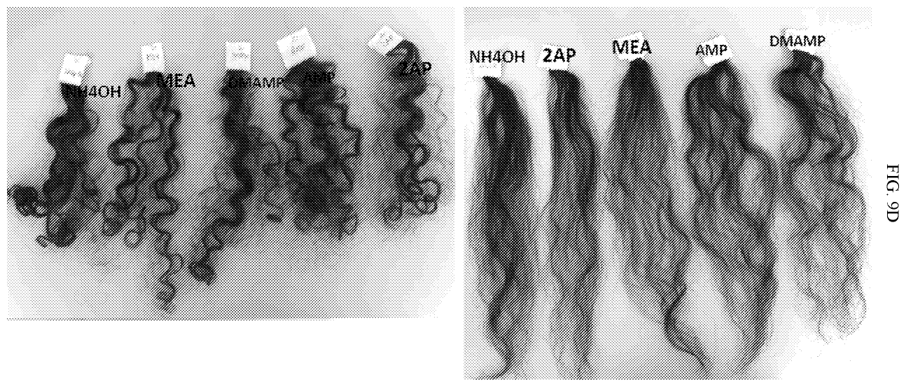

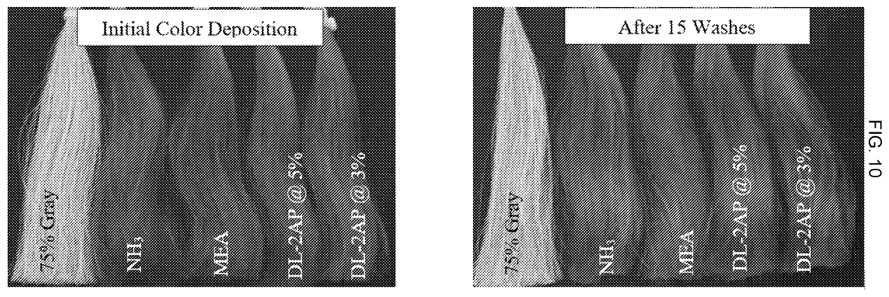

All components in Phase A were mixed and heated to 75° C. to provide Phase A. All components in Phase B were mixed and heated to 75° C. to provide Phase B. Under stirring Phase A was poured into Phase B and stirring was continued for 10 minutes. The temperature was reduced to 40° C. and the component of phase C were added under stirring to provide composition 1. Composition 2 was added to composition 1 under stirring to provide the compositions containing the alkaline agent ammonia, DL-2AP, MEA, AMP, or AMPD (Table 10). Each straightening composition was applied to virgin hair samples by brushing onto the hair. After 30 minutes at room temperature, the straightening compositions were washed from the hair samples with tap water until clean and the hair was air dried to provide the curl-altered hair samples (FIGS. 9A and 9B). A flat iron was applied to each dried hair sample 3-times and a 20 vol % hydrogen peroxide solution as applied, while keeping the hair straight. After 5 minutes, the solution was washed from the hair samples and the hair was air dried to provide the curl-altered hair samples (FIGS. 9C and 9D).

TABLE 10
Straightening Compositions

| | | | | | |
|---|---|---|---|---|---|
| | Ammonia | DL-2AP | MEA | AMP | AMPD |
| | Compo- | Compo- | Compo- | Compo- | Compo- |
| | sition | sition | sition | sition | sition |
| Component | (wt %) | (wt %) | (wt %) | (wt %) | (wt %) |

Composition 1
Phase A

| | | | | | |
|---|---|---|---|---|---|
| Water | 44.5 | 44.5 | 44.5 | 44.5 | 44.5 |
| Tetrasodium | 0.1 | 0.1 | 0.1 | 0.1 | 0.1 |
| EDTA | | | | | |
| Cetrimonium | 1.5 | 1.5 | 1.5 | 1.5 | 1.5 |
| chloride | | | | | |
| Glycerin | 1.0 | 1.0 | 1.0 | 1.0 | 1.0 |
| Propylene | 1.0 | 1.0 | 1.0 | 1.0 | 1.0 |
| glycol | | | | | |

Phase B

| | | | | | |
|---|---|---|---|---|---|
| Cetearyl | 15 | 15 | 15 | 15 | 15 |
| alcohol | | | | | |
| (Vegarol 1618) | | | | | |
| Ceteareth-20 | 3.0 | 3.0 | 3.0 | 3.0 | 3.0 |
| (Cetearyl | | | | | |
| alcohol | | | | | |
| ethoxylate, 20 | | | | | |
| EO) | | | | | |
| Steareth-20 | 3.5 | 3.5 | 3.5 | 3.5 | 3.5 |
| (Lipocol S-20) | | | | | |

Phase C

| | | | | | |
|---|---|---|---|---|---|
| Thioglycolic | 8 | 8 | 8 | 8 | 8 |
| acid (99%) | | | | | |
| Ammonia (28- | 11 | — | — | — | — |
| 30%) | | | | | |
| DL-2AP | — | 9 | — | — | — |
| MEA | — | — | 8 | — | — |
| AMP | — | — | — | 11 | — |
| AMPD | — | — | — | — | 15 |

Composition 2

| | | | | | |
|---|---|---|---|---|---|
| Water | 10.4 | 10.4 | 10.4 | 10.4 | 10.4 |
| Ammonium | 2 | 2 | 2 | 2 | 2 |
| bicarbonate | | | | | |

Properties

| | | | | | |
|---|---|---|---|---|---|
| pH | 9.3 | 9.02 | 9.01 | 9.05 | 8.7 |

### Example 5. Gray Hair Coverage

Permanent hair color is popular for use to cover grays. For such applications it is important for a hair color formula to provide color lift on darker hair, along with color deposition on all hair. This study was conducted using 75% gray hair to allow for comparison of performance on color lift for darker hair and subsequent color deposition to provide a uniform end color shade. DL-2AP was tested at 5% and 3% usage in comparison with ammonia and MEA. The effects of permanent color development and fastness can be seen in FIG. 10. All systems demonstrated comparable color fastness.

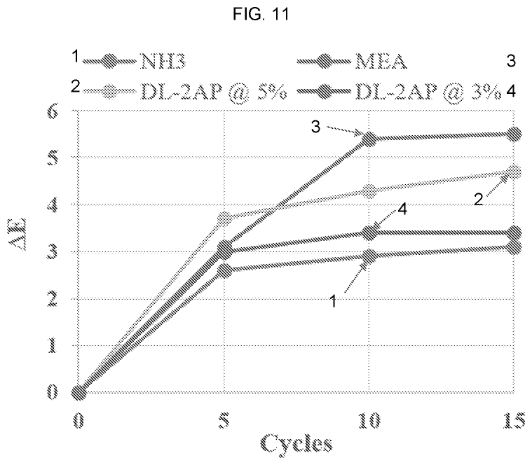

Table 11 exhibits the gray hair color deposition and DL-2AP showed comparable color change as measured by ΔE vs. NH₃ and MEA. FIG. 11 shows color retention for two levels of DL-2AP.

TABLE 11
Gray Coverage Hair Deposition (1 Cycle)

| | ΔE | Color Parameters | | |
|---|---|---|---|---|
| | 1 Treatment | L | a | b |
| NH₃ | 17.4 | 54.4 | 14.5 | 21.8 |
| MEA | 16.7 | 55.6 | 15.0 | 20.8 |
| DL-2AP 5% | 14.6 | 56.9 | 13.5 | 20.0 |
| DL-2AP 3% | 17.4 | 54.4 | 14.5 | 21.8 |

### Equivalents

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A hair-altering composition comprising an alkaline agent comprising 2-aminopropanol.
B. The hair-altering composition of paragraph A, wherein the 2-aminopropanol is present in an amount of about 2 wt % to about 15 wt %.
C. The hair-altering composition of paragraph A or paragraph B, wherein the 2-aminopropanol is present in an amount of about 3 wt % to about 14 wt %.
D. The hair-altering composition of any one of paragraphs A-C, wherein the 2-aminopropanol is present in an amount of about 4 wt % to about 12 wt %.
E. The hair-altering composition of any one of paragraphs A-C, wherein the 2-aminopropanol is present in an amount of about 3 wt % to about 10 wt %.
F. The hair-altering composition of any one of paragraphs A-E, wherein the 2-aminopropanol is present in an amount of about 6 wt % to about 11 wt %.
G. The hair-altering composition of any one of paragraphs A-F further comprising ammonia, an alkanolamine different from 2-aminopropanol, or a combination thereof.
H. The hair-altering composition paragraph G, wherein the alkanolamine comprises 2-amino-2-methyl-1-propanol (AMP), 2-dimethylamino-2-methyl-1-propanol (DMAMP), 2-amino-1-butanol (AB), 2-amino-2-methyl-1,3-propanediol (AMPD), 2-amino-2-ethyl-1,3-propandiol (AEPD), Tris(hydroxymethyl)aminomethane (TA), monoethanolamine (MEA), monoisopropanolamine (MIPA), Triethanolamine (TEA) Triisopropanolamine (TIPA), or combinations of two or more thereof.
I. The hair-altering composition of any one of paragraphs A-F, wherein the composition is substantially free of ammonia.
J. The hair-altering composition of any one of paragraphs A-F or I, wherein the composition is substantially free of MEA.
K. The hair-altering composition of any one of paragraphs A-J further comprising a coloring agent.
L. The hair-altering composition paragraph K, wherein the coloring agent comprises anion dyes, cation dyes, neutral dyes, or combinations of two or more thereof.
M. The hair-altering composition of any one of paragraphs A-L further comprising an antioxidant.
N. The hair-altering composition paragraph M, wherein the antioxidant comprises ascorbic acid, sodium sulfite, sodium metabisulfite, sodium hydrosulfite, tocopherols, α-tocopherol, tocopheryl acetate, panthenol, selenium sulfide, zinc formosulfoxylate, isoascorbic acid, magnesium ascorbyl phosphate, ascorbyl glucoside, cysteine, thiourea, glyceryl monothioglycolate, thioglycerol, 2,5-dihydroxybenzoic acid, erythorbic acid, or combinations of two or more thereof.
O. The hair-altering composition of any one of paragraphs A-N further comprising one or more surface active agents.
P. The hair-altering composition paragraph O, wherein the one or more surface active agents comprise cocamidopropyl betaine, PEG-40 hydrogenated castor oil, sodium lauryl sulfate, sodium laureth sulfate, Oleth-20, glyceryl stearate, PEG-40 stearate, ceteareth-20, or combinations of two or more thereof.
Q. The hair-altering composition of any one of paragraphs A-P further comprising an alcohol.
R. The hair-altering composition paragraph Q, wherein the alcohol comprises linear saturated C₁₄-C₂₀ alcohol, linear unsaturated C₁₄-C₂₀ alcohol, or a combination thereof.
S. The hair-altering composition paragraph Q or paragraph R, wherein the alcohol comprises cetearyl alcohol, oleyl alcohol, cetyl alcohol, stearyl alcohol, behenyl alcohol or a combination thereof.
T. The hair-altering composition of any one of paragraphs A-S further comprising a skin conditioning agent.
U. The hair-altering composition of any one of paragraphs A-T further comprising a fragrance.
V. The hair-altering composition of any one of paragraphs A-T further comprising a curl modifying agent.
W. The hair-altering composition of paragraph V, wherein the curl modifying agent comprises an alkaline straightener, thioglycolate, keratin, or combinations of two or more thereof.
X. The hair-altering composition of paragraph V or paragraph W, wherein the curl modifying agent comprises thioglycolate.
Y. The hair-altering composition of any one of paragraphs A-X further comprising a developer.
Z. The hair-altering composition of paragraph Y, wherein the developer comprises hydrogen peroxide.
AA. The hair-altering composition of any one of paragraphs A-Z further comprising water.
BB. The hair-altering composition of any one of paragraphs A-AA, wherein the hair-altering composition is a hair color altering composition.
CC. The hair-altering composition of paragraph BB, wherein hair treated with the composition exhibits a hair color altering ΔE of less than about 2.5 compared to a control composition having ammonia substituted for the 2-aminopropanol.
DD. The hair-altering composition of paragraph BB or paragraph CC, wherein hair treated with the composition exhibits a hair color altering ΔE of less than about 2 compared to a control composition having ammonia substituted for the 2-aminopropanol.
EE. The hair-altering composition of paragraph CC or paragraph DD, wherein the ΔE is measured after one or more color treatment cycles.
FF. The hair-altering composition of paragraph EE, wherein the ΔE is measured after five or more color treatment cycles.
GG. The hair-altering composition of any one of paragraphs A-FF, wherein the hair-altering composition is a curl, kink, or hair texture altering composition.
HH. The hair-altering composition of paragraph GG, wherein the hair-altering composition is a disulfide bond breaking composition.
II. A method for altering hair comprising:
   applying the hair altering composition of any one of paragraphs A-HH on a hair; and
   rinsing the composition off of the hair with water to provide the altered hair.
JJ. The method of paragraph II, wherein the rinsing occurs at least about 10 minutes after the applying.
KK. The method of paragraph II or paragraph JJ, wherein the rinsing occurs at least about 20 minutes after the applying.
LL. The method of paragraph II or paragraph JJ, wherein the rinsing occurs at least about 30 minutes after the applying.
MM. The method of any one of paragraphs II-LL, wherein the composition is a color altering composition and the altered hair exhibits a hair color altering ΔE of less than about 2.5 compared to a hair treated with a control composition having ammonia substituted for the 2-aminopropanol.
NN. The method of any one of paragraphs II-MM, wherein the composition is a color altering composition and the altered hair exhibits a hair color altering ΔE of less than about 2 compared to a hair treated with a control composition having ammonia substituted for the 2-aminopropanol.
OO. The method of paragraph MM or paragraph NN, wherein the ΔE is measured after applying and rinsing one or more times.
PP. The method of paragraph MM or paragraph NN, wherein the ΔE is measured after applying and rinsing five or more times.
QQ. The method of any one of paragraphs II-PP, wherein the composition is a curl altering composition.
RR. A kit comprising the hair altering composition of any one of paragraphs A-HH.
SS. The kit of paragraph RR, wherein the kit comprises a first composition comprising the 2-aminopropanol and a second composition comprising the developer.

1. A hair-altering composition comprising an alkaline agent comprising 2-aminopropanol.

2. The hair-altering composition of claim 1, wherein the 2-aminopropanol is present in an amount of about 2 wt % to about 15 wt %.

3. The hair-altering composition of claim 1, further comprising ammonia, an alkanolamine different from 2-aminopropanol, or a combination thereof.

4. The hair-altering composition claim 3, wherein the alkanolamine comprises 2-amino-2-methyl-1-propanol (AMP), 2-dimethylamino-2-methyl-1-propanol (DMAMP), 2-amino-1-butanol (AB), 2-amino-2-methyl-1,3-propanediol (AMPD), 2-amino-2-ethyl-1,3-propandiol (AEPD), Tris(hydroxymethyl)aminomethane (TA), monoethanolamine (MEA), monoisopropanolamine (MIPA), Triethanolamine (TEA) Triisopropanolamine (TIPA), or combinations of two or more thereof.

5. The hair-altering composition of claim 1, wherein the composition is substantially free of ammonia and/or substantially free of MEA.

6. The hair-altering composition of claim 1 further comprising a coloring agent, wherein the coloring agent comprises anion dyes, cation dyes, neutral dyes, or combinations of two or more thereof.

7. The hair-altering composition of claim 1 further comprising one or more of:
an antioxidant,
a surface active agent,
an alcohol,
a skin conditioning agent and/or a fragrance,
a curl modifying agent,
a developer, or
water.

8. The hair-altering composition claim 7, wherein the antioxidant comprises ascorbic acid, sodium sulfite, sodium metabisulfite, sodium hydrosulfite, tocopherols, α-tocopherol, tocopheryl acetate, panthenol, selenium sulfide, zinc formosulfoxylate, isoascorbic acid, magnesium ascorbyl phosphate, ascorbyl glucoside, cysteine, thiourea, glyceryl monothioglycolate, thioglycerol, 2,5-dihydroxybenzoic acid, erythorbic acid, or combinations of two or more thereof.

9. (canceled)

10. The hair-altering composition claim 7, wherein the one or more surface active agents comprise cocamidopropyl betaine, PEG-40 hydrogenated castor oil, sodium lauryl sulfate, sodium laureth sulfate, Oleth-20, glyceryl stearate, PEG-40 stearate, ceteareth-20, or combinations of two or more thereof.

11. The hair-altering composition of claim 7 wherein the alcohol comprises linear saturated C₁₄-C₂₀ alcohol, linear unsaturated C₁₄-C₂₀ alcohol, or a combination thereof.

12. The hair-altering composition claim 11, wherein the alcohol comprises cetearyl alcohol, oleyl alcohol, cetyl alcohol, stearyl alcohol, behenyl alcohol or a combination thereof.

13. (canceled)

14. (canceled)

15. The hair-altering composition of claim 7, wherein the curl modifying agent comprises an alkaline straightener, thioglycolate, keratin, or combinations of two or more thereof.

16. (canceled)

17. The hair-altering composition of claim 7, wherein the developer comprises hydrogen peroxide.

18. (canceled)

19. The hair-altering composition of claim 1, wherein the hair-altering composition is one or more of:
a hair color altering composition,
a curl, kink, or hair texture altering composition, and
a disulfide bond breaking composition.

20. The hair-altering composition of claim 19, wherein hair treated with the composition exhibits a hair color altering ΔE of less than about 2.5 compared to a control composition having ammonia substituted for the 2-aminopropanol.

21. The hair-altering composition of claim 20, wherein the ΔE is measured after one or more color treatment cycles.

22. (canceled)

23. (canceled)

24. A method for altering hair comprising:
applying the hair altering composition of claim 1 on a hair; and
rinsing the composition off of the hair with water to provide the altered hair,
optionally wherein the rinsing occurs at least about 10 minutes after the applying.

25. (canceled)

26. The method of claim 24, wherein the composition is a color altering composition and the altered hair exhibits a hair color altering ΔE of less than about 2.5 compared to a hair treated with a control composition having ammonia substituted for the 2-aminopropanol.

27. The method of claim 26, wherein the ΔE is measured after applying and rinsing one or more times.

28. The method of claim 24, wherein the composition is a curl altering composition.

29. (canceled)
